(19) United States
(12) Patent Application Publication
(10) Pub. No.: US 2026/0038116 A1
(43) Pub. Date: Feb. 5, 2026

(54) METHOD AND APPARATUS FOR USING DIGITIZED IMAGING DATA TO AID PATIENT MANAGEMENT

(71) Applicants: Case Western Reserve University, Cleveland, OH (US); University Hospitals Cleveland Medical Center, Cleveland, OH (US); The Methodist Hospital System, Houston, TX (US)

(72) Inventors: David L. Wilson, Cleveland Heights, OH (US); Ammar Hoori, Westlake, OH (US); Hao Wu, Cleveland, OH (US); Robert C. Gilkeson, Cleveland, OH (US); Sanjay Rajagopalan, Cleveland, OH (US); Sadeer Al-Kindi, Manvel, TX (US); Juhwan Lee, Westlake, OH (US)

(21) Appl. No.: 19/288,220

(22) Filed: Aug. 1, 2025

Related U.S. Application Data
(60) Provisional application No. 63/678,568, filed on Aug. 2, 2024.
(60) Provisional application No. 63/847,586, filed on Jul. 21, 2025.

(51) Int. Cl. G06T 7/00; A61B 5/318; G06T 7/11; G16H 15/00; G16H 30/40; G16H 50/20

(52) U.S. Cl. CPC G06T 7/0012; A61B 5/318; G06T 7/11; G16H 15/00; G16H 30/40; G16H 50/20; G06T 2207/10081; G06T 2207/30048; G06T 2207/30104

(57) ABSTRACT

In some embodiments, the present disclosure relates to a method that includes accessing data stored in an electronic memory. The data includes digitized imaging data from a segmented non-contrast computerized tomography (CT) image of an obstructive coronary artery disease (OCAD) patient. A plurality of assessment features are extracted from the data. The plurality of assessment features include image based features that characterize one or more of calcifications, fat tissue, heart structures, bone density, muscle, a lung, and breast tissue. The plurality of assessment features and a plurality of clinical factors are provided to a machine learning stage that is configured to generate a medical assessment corresponding to whether or not the OCAD patient would benefit from additional diagnostic tests to identify a presence and extent of the obstructive coronary artery disease.

### REFERENCE TO RELATED APPLICATIONS

This application claims the benefit of U.S. Provisional Application No. 63/847,586, filed on Jul. 21, 2025 & U.S. Provisional Application No. 63/678,568, filed on Aug. 2, 2024. The contents of the above-referenced Patent Applications are hereby incorporated by reference in their entirety.

### FEDERAL FUNDING INFORMATION

This invention was made with government support under HL165218 and HL167199 awarded by the National Institutes of Health. The government has certain rights in the invention.

### BACKGROUND

The heart is a fist-sized organ that pumps blood with oxygen and nutrients to different parts of the human body through a network of blood vessels. The heart is the primary organ of a circulatory system and is essential for human life. However, over time cardiovascular disease can cause damage to a heart and lead to events such as strokes, myocardial infractions, and/or even death.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of a cardiovascular assessment system comprising a machine learning stage configured to use assessment features to generate a medical assessment.

FIG. 2 illustrates a flow diagram showing some embodiments of a method of operating a machine learning model on assessment features to generate a medical assessment.

FIG. 3 illustrates some additional embodiments of a cardiovascular assessment system comprising a machine learning stage configured to generate a medical assessment using assessment features extracted from digitized imaging data and electrocardiogram data.

FIG. 4 illustrates a flow diagram showing some embodiments of a method of operating a machine learning model to generate a medical assessment using assessment features extracted from digitized imaging data and electrocardiogram data.

FIG. 5 illustrates some embodiments of a graph showing a likelihood of a major adverse cardiovascular event (MACE) occurring as a function of time generated by a model using both image assessment features and EKG assessment features.

FIG. 6 illustrates some additional embodiments of a cardiovascular assessment system comprising a machine learning stage configured to generate a medical assessment.

FIG. 7 illustrates an example receiver operating characteristic (ROC) curve corresponding to a risk of ischemia generated by different models.

FIG. 8 illustrates some additional embodiments of a cardiovascular assessment system comprising a machine learning stage configured to generate a medical assessment.

FIG. 9 illustrates some additional embodiments of a cardiovascular assessment system comprising a machine learning stage configured to generate a medical assessment.

FIG. 10 illustrates some embodiments of a block diagram showing operation of a disclosed cardiovascular assessment system.

FIG. 11 illustrates some additional embodiments of a cardiovascular assessment system comprising a machine learning stage configured to generate a medical assessment.

FIG. 12 illustrates a flow diagram showing some additional embodiments of a method of operating a machine learning stage on assessment features to generate a medical assessment.

FIG. 13 illustrates a block diagram of some embodiments of a cardiovascular assessment system comprising a machine learning stage configured to generate a medical assessment.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Heart disease refers to any problem affecting the heart, such as coronary artery disease, arrhythmia, heart failure, and/or the like. According to the Centers for Disease Control and Prevention (CDC), heart disease is the leading cause of death in the United States and is responsible for about 1 in 4 deaths. Coronary artery disease, also known as ischemic heart disease or myocardial ischemia, is the most common type of heart disease. It develops when the arteries that supply blood to the heart become clogged with plaque. This causes them to harden and narrow. Plaque contains cholesterol and other substances. Over time, coronary artery disease can lead to a heart attack, abnormal heart rhythms, or heart failure.

In assessing patients for coronary artery disease, clinicians currently consider a host of clinical data (e.g., symptoms, electrocardiograms (ECGs), blood tests, blood pressure history, etc.) and whole-heart Agatston score from computed tomography calcium score (CTCS) images to determine future, long-term risk and to guide pharmacotherapies. However, once coronary calcifications are identified, there is no consensus on which patients should get additional tests to identify or rule out obstructive coronary artery disease. This is important as many patient groups may benefit from revascularization (e.g. stents or bypass surgery) to improve outcomes beyond medications alone.

Multiple potential diagnostic choices exist, including anatomic (e.g., coronary computed tomography angiography (CCTA), x-ray angiography, etc.) and functional (e.g., stress ECG, pharmacologic or exercise SPECT and PET, pharmacologic stress MRI, etc.) choices. CCTA is emerging as a more common choice because it alone can give a non-invasive, detailed assessment of coronary artery disease morphology. Moreover, computationally detailed evaluations of CCTA images are clinically approved and emerging, including fractional flow reserve (FFR) assessments from vessel narrowing, analyses of blood vessel high-risk features, and analyses of pericoronary adipose tissue.

Currently, the decision to move to additional workup and the choice of a test (e.g., anatomic vs functional) often includes consideration of a whole-heart Agatston score. However, while the whole heart Agatston score may be a surrogate for atherosclerosis burden, it does not explicitly provide information about coronary stenoses leading to myocardial ischemia. Because of this, it has been appreciated that a whole heart Agatston score may not provide for high accuracy of diagnosis. In clinical practice only 3% to 6% of patients identified for additional testing by Agatston score turn out to be ischemia-positive, making it unclear which patients with elevated Agatston scores will benefit from additional ischemia imaging (e.g., CCTA). This is a significant problem since making the correct decision for additional diagnostic imaging may avoid unnecessary testing that can be costly, associated with risks (e.g., radiation exposure), and drain resources (e.g., imaging systems, technical staffing, and physicians).

The present disclosure relates to a cardiovascular assessment method and apparatus that utilizes assessment features extracted from a digitized image (e.g., a non-contrast computed tomography (CT) image of the heart, including a gated CT calcium score image, a non-gated lung cancer screening image, or the like) to provide a personalized assessment of patients that would benefit from further coronary evaluation analysis. In some embodiments, the cardiovascular assessment method comprises accessing data corresponding to an obstructive coronary artery disease (OCAD) patient from an electronic memory. A plurality of assessment features are extracted from the data. The plurality of assessment features may include image assessment features. A machine learning model is operated upon the plurality of assessment features and a plurality of clinical factors to generate a medical assessment corresponding to whether or not the OCAD patient would benefit from additional diagnostic tests to identify a presence and/or extent of obstructive coronary artery disease. The generation of the medical assessment using assessment features extracted from digitized imaging data allows the disclosed method and apparatus to make a highly accurate prediction at a low-cost and low risk to the OCAD patient.

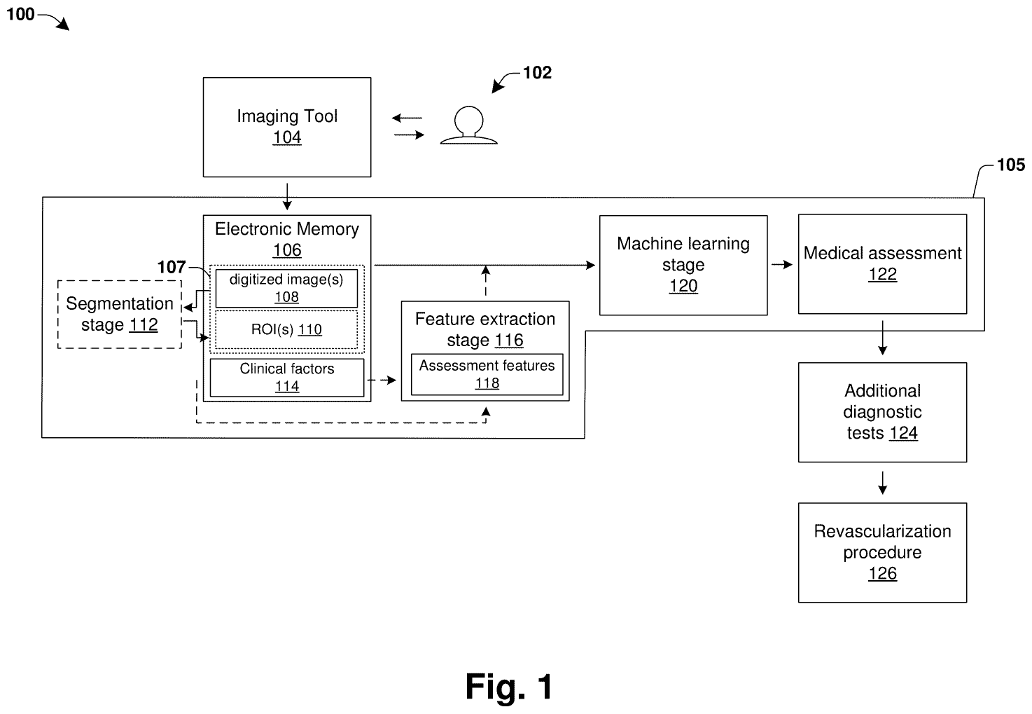

FIG. 1 illustrates some embodiments of a cardiovascular assessment system 100 comprising a machine learning stage configured to use assessment features to generate a medical assessment.

The cardiovascular assessment system 100 comprises a cardiovascular assessment apparatus 105 that includes an electronic memory 106 configured to store data 107 relating to an obstructive coronary artery disease (OCAD) patient 102. In some embodiments, the data 107 may comprise digitized imaging data that includes one or more digitized images 108 of the OCAD patient 102. The one or more digitized images 108 may include a computed tomography (CT) image, a non-contrast computed tomography (CT) image, a computed tomography angiography (CTA) image, a computed tomography calcium score (CTCS) image of the patient's chest (e.g., a CT calcium score image, a lung cancer screening image), a gated CT image (e.g., a gated CT calcium score image), a non-gated CT image (e.g., a non-gated lung cancer screening image), a photon counting CT (PCCT) image, and/or the like. In some embodiments, the digitized imaging data may be obtained from an imaging tool 104 that is configured to operate upon the OCAD patient 102. The imaging tool 104 may comprise a CT machine with an integrating detector, an energy sensitive CT machine (e.g., using multiple types of implementations), a photon-counting CT machine, and/or the like. In some additional embodiments, the electronic memory 106 may be further configured to store one or more clinical factors 114 relating to the OCAD patient 102.

In some embodiments, a segmentation stage 112 is in communication with the electronic memory 101. The segmentation stage 112 is configured to segment the one or more digitized images 108 to generate one or more segmented digitized images that respectively identify one or more regions of interest (ROI) 110 (e.g., volumes of interest). In some embodiments, the one or more ROI 110 may comprise calcifications, heart structures, adipose tissue, and/or the like. In some embodiments, the segmented digitized images and/or the one or more ROI 110 may be stored in the electronic memory 101 as part of the data 107.

A feature extraction stage 116 is configured to extract a plurality of assessment features 118 from the data 107 (e.g., from the one or more ROI 110). The plurality of assessment features 118 include features that have been found to be prognostic of obstructive coronary artery disease, atherosclerotic events, and/or ischemia. For example, the plurality of assessment features 118 may include features that characterize the one or more ROI 110. For example, the plurality of assessment features 118 may characterize one or more of calcifications (e.g., calcium-omics), adipose tissue (e.g., fat-omics), pericoronary adipose tissue (e.g., PCAT-omics). In some embodiments, the plurality of assessment features 118 may additionally and/or alternatively include features that characterize heart structures, bone density, muscle, lungs, breast tissue, etc. The plurality of assessment features 118 may include and/or be numeric values (e.g., a calcification mass score of an aortic value, a volume of epicardial fat, etc.), probabilities of existing disease (e.g., a probability of fat inflammation, a probability of nonalcoholic fatty liver disease (NAFLD), etc.), and/or the like.

A machine learning stage 120 is configured to utilize the plurality of assessment features 118 and the one or more clinical factors 114 to generate a medical assessment 122. In some embodiments, the medical assessment 122 provides advice as to whether or not the OCAD patient 102 would benefit from using additional diagnostic tests (e.g., additional diagnostic imaging) to identify a presence and/or extent of obstructive coronary artery disease. In other embodiments, the medical assessment 122 may additionally or alternatively provide for an assessment of atherosclerotic disease, disease probabilities (e.g., a probability of fat inflammation, NAFLD, etc.), a risk prediction (e.g., a risk of a coronary event within a predetermined time period of 10 years), and/or the like.

In some embodiments, depending upon the medical assessment 122, one or more additional diagnostic tests 124 may be performed on the OCAD patient 102 to determine whether or not a revascularization procedure 126 would be beneficial for the OCAD patient 102. In some embodiments, the one or more additional diagnostic tests 124 may comprise a positron emission tomography (PET) scan, a magneto resonance imaging (MRI) scan, a single-photon emission computed tomography (SPECT) scan, a positron emission tomography (PET) scan, and/or the like. In some embodiments, the additional diagnostic tests 124 may comprise anatomic tests (e.g., x-ray angiography) and/or functional tests (e.g., stress ECG, pharmacologic or exercise SPECT and PET, and pharmacologic stress MRI). In some embodiments, the one or more additional diagnostic tests 124 may comprise a cardiovascular computed tomography angiography (CCTA). The CCTA is configured to provide for a non-invasive, detailed assessment of coronary artery disease morphology. In some embodiments, the one or more additional diagnostic tests 124 may comprise a CCTA including fraction flow reserve (FFR) assessments from vessel narrowing, analyses of blood vessel high-risk features, and/or analyses of pericoronary adipose tissue.

It has been appreciated that generating the medical assessment 122 by operating the machine learning stage 120 on the plurality of assessment features 118 and the one or more clinical factors 114 allows for the medical assessment 122 to be more accurate than previous assessment methods (e.g., a whole heart Agatston score). Furthermore, generating the medical assessment 122 by operating the machine learning stage 120 on the plurality of assessment features 118 extracted from other imaging data (e.g., lower cost and/or more benign imaging data) can offer advantages. For example, CT scans are low-cost and low-radiation scans that are widely used for early detection of atherosclerosis. By using CT images to determine whether more expensive and/or evasive additional diagnostic testing is warranted, medical professionals can use low-cost scans that are already in common practice to make more informed decisions that can avoid subjecting the OCAD patient 102 to unnecessary costs and/or risks (e.g., due to radiation). Moreover, CT imaging is widely available therefore making the disclosed medical assessment available to many vulnerable and underserved populations having significant barriers to healthcare. Although the disclosed medical assessment 122 is described above as determining whether or not a revascularization procedure 126 would be beneficial for the OCAD patient 102, it will be appreciated that the disclosed method and apparatus may be applied to other areas where an imaging study can be used to decide on more advanced imaging (e.g., mammography versus breast MRI, sonography versus CT/MRI/PET, etc.).

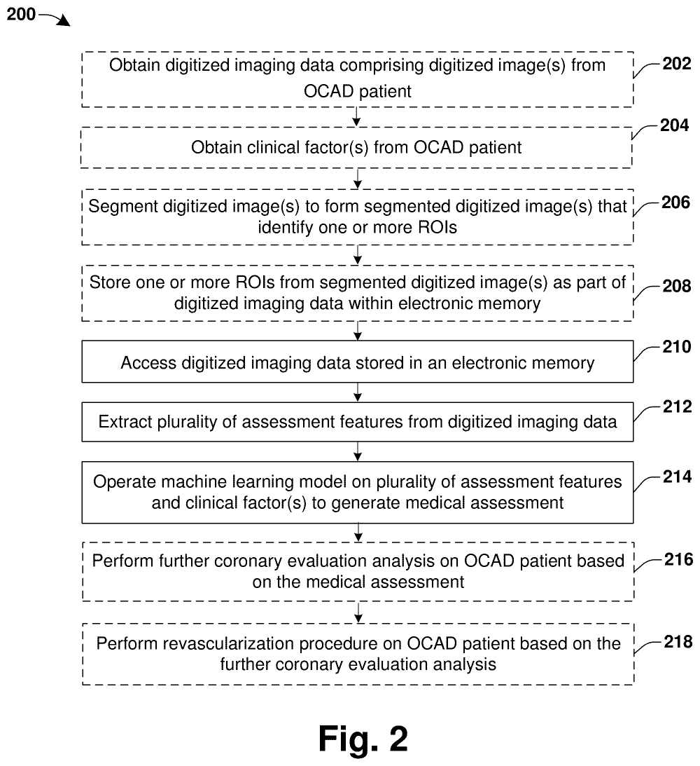

FIG. 2 illustrates a flow diagram showing some embodiments of a method 200 of operating a machine learning stage on assessment features to generate a medical assessment.

While the disclosed methods (e.g., method 200, 400, and/or 800) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 202, digitized imaging data comprising one or more digitized images from a patient is obtained. In some embodiments, the patient may be an OCAD (obstructive coronary artery disease) patient. In some embodiments, the digitized imaging data may comprise a computed tomography (CT) image, a non-contrast computed tomography (CT) image, a computed tomography angiography (CTA) image, a computed tomography calcium score (CTCS) image of the patient's chest, a non-contrast low-dose computed tomography calcium score (CTCS) image of the patient, a gated CT image (e.g., a gated CT calcium score image), a non-gated CT image (e.g., a non-gated lung cancer screening image), a photon counting CT (PCCT) image, and/or the like. The digitized imaging data may be obtained by placing the patient in an imaging device (e.g., on a table of a computer tomography (CT) machine and then moving the patient into a gantry of the CT machine) and then operating the imaging device on the patient.

At act 204, one or more clinical factors are obtained from the patient. In some embodiments, the one or more clinical factors may include a blood pressure, chest pain, lipid assessments, presence of comorbidities such as diabetes and psoriasis, high cholesterol, obesity, a body mass index (BMI), and/or the like. The one or more clinical factors may be obtained by physically acting upon the patient. For example, a blood pressure may be obtained by having a health care professional wrap an inflatable cuff around the patient, while lipid assessment may be obtained by drawing blood from the patient.

At act 206, the one or more digitized images may be segmented to form one or more segmented digitized images that identify one or more regions of interest (ROIs). In some embodiments, the one or more ROIs may comprise one or more of a heart, calcifications (e.g., coronary calcification, aortic calcifications, aortic valve calcifications, mitral annulus calcifications, or the like), fat tissue (e.g., epicardial fat, liver fat, pericardial fat, subcutaneous fat, periaortic fat, pericoronary fat, and/or the like), heart structures (e.g., whole heart, right atrium, right ventricle, left atrium, left ventricle, aortic root, etc.), bone (e.g., spin thoracic vertebrae, etc.), muscle (e.g., skeletal muscle, pectoralis muscle, etc.), a lung, breast tissue, and/or the like. In some embodiments, the one or more digitized images may be automatically segmented using one or more deep learning models.

At act 208, the one or more ROIs may be stored as part of the digitized imaging data within an electronic memory, in some embodiments.

At act 210, the digitized imaging data is accessed from the electronic memory.

At act 212, a plurality of assessment features are extracted from the one or more regions of interest within the digitized imaging data.

At act 214, a machine learning model is operated on the plurality of assessment features and the one or more clinical factors to generate a medical assessment. In some embodiments, the medical assessment may be for additional diagnostic testing to identify obstructive coronary artery disease. The additional guidance will help health care professionals decide if the patient would benefit from additional diagnostic tests to identify a presence and extent of obstructive coronary artery disease.

At act 216, further coronary evaluation analysis may be performed on the patient based upon the medical assessment. The further coronary evaluation analysis may include anatomic testing (e.g., CCTA, x-ray angiography, etc.) and/or functional testing (e.g., stress ECG, pharmacological or exercise SPECT and PET, pharmacological stress MRI, etc.).

At act 218, a revascularization procedure may be performed on the patient based upon the further coronary evaluation analysis. The revascularization procedure may include placement of a stent, a bypass surgery, an endarterectomy, and/or the like.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

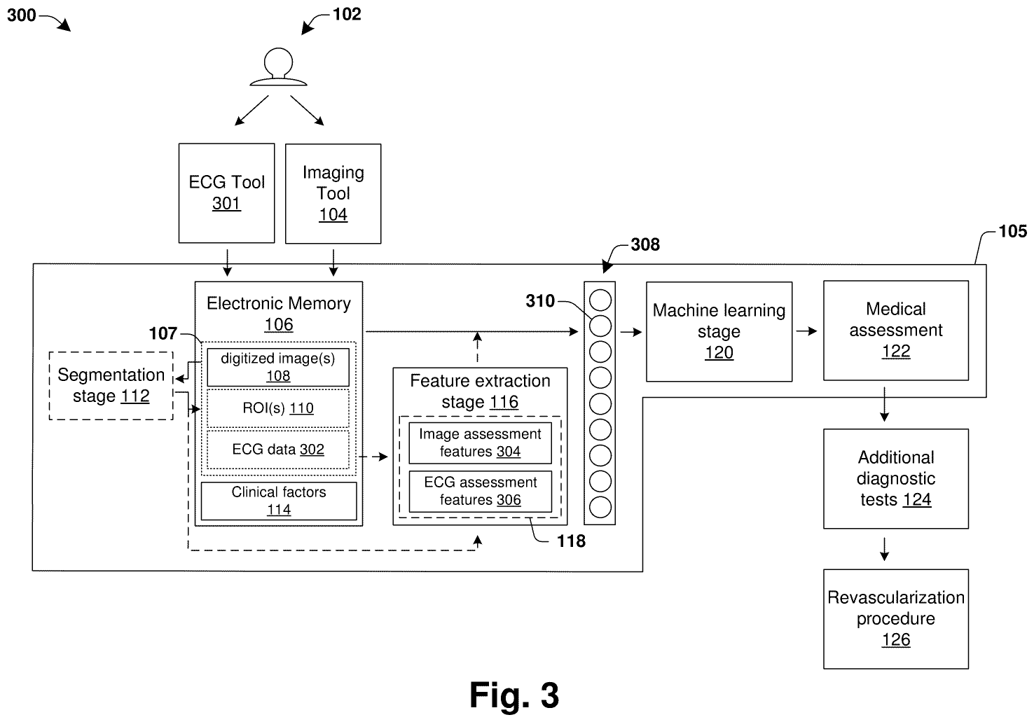

FIG. 3 illustrates some additional embodiments of a cardiovascular assessment system 300 comprising a machine learning stage configured to generate a medical assessment.

The cardiovascular assessment system 300 comprises an electronic memory 106 configured to store data 107 for an OCAD patient 102. In some embodiments, the data 107 may comprise digitized imaging data that includes one or more digitized images 108 and/or one or more ROI 110 from the one or more digitized images 108. In some embodiments, the electronic memory 106 may further store one or more clinical factors 114 relating to the OCAD patient 102. The one or more clinical factors 114 may include a body mass index (BMI), a blood pressure (BP), a lipid status (e.g., yes/no), a diabetes status, etc.

In some embodiments, the data 107 may further comprise electrocardiogram (ECG) data 302 relating to the OCAD patient 102. The ECG data 302 represents the electrical activity of the heart of the OCAD patient 102. The ECG data 302 may be recorded using sensors attached to the skin of the OCAD patient 102 and provides information about the heart's rhythm and rate. The ECG data 302 may include one or more of P wave data (corresponding to atrial contraction), QRS complex data (corresponding to ventricular contraction), T wave data (corresponding to ventricular relaxation), PR interval data (corresponding to a time for electrical activity to travel from atria to ventricles), QT interval data (corresponding to a time for ventricles to contract and relax), ST segment data (corresponding to a time between ventricular contraction and relaxation), and/or the like.

In some embodiments, the ECG data 302 may be collected by operating an ECG tool 301 on the OCAD patient 102. The ECG tool 301 comprises a plurality of electrodes that are attachable to the OCAD patient 102. The plurality of electrodes may comprise 3 electrodes, 10 electrodes, or another number of electrodes. Electrode signals may be combined to create leads (e.g., 1 lead, 3 leads, 12 leads, and/or the like). In some embodiments, the ECG data 302 may be collected in a manner that is synchronized with collection of the digitized imaging data (e.g., the imaging may be ECG gated).

A feature extraction stage 116 is configured to extract a plurality of assessment features 118 that have been found to be prognostic of obstructive coronary artery disease and/or ischemia from the data 107. The plurality of assessment features 118 include image based assessment features 304 and/or ECG based assessment features 306. The image based assessment features 304 are extracted from the digitized imaging data (e.g., from the one or more ROI 110). In some embodiments, the image based assessment features 306 may include features that characterize calcifications and adipose tissue. The ECG based assessment features 306 are extracted from the ECG data 302.

The image based assessment features 304 that characterize calcifications may include a number of calcifications, a number of territories, a mass, a volume, an average of Hounsfield units (HU), a peak HU, a mean HU, a median HU, a spatial spread [diffusivity], a distance between coronary artery calcifications, topological features (e.g., surface area, diameter, center of mass, etc.) in two dimensions (2D) and three dimensions (3D), a number and connectedness of calcifications at varying HU thresholds, etc. In some embodiments, image based assessment features 304 that characterize one or more calcifications may be aggregated over a whole heart, an artery, and/or an individual calcification. For example, the features may include a left circumflex artery mass score, a number of coronary artery calcifications in the left Anterior Descending (LAD) artery, a mean value of HU measured in calcifications, a HU histogram for a left anterior descending artery, a total heart mass score, etc.

The plurality of image based assessment features 304 that characterize adipose tissue may include features that characterize pericoronary adipose tissue, epicardial adipose tissue, and/or intramyocardial adipose tissue (e.g., volume, spatial distribution, regional fat volumes associated with chambers, thicknesses in regions, thickness histograms, assessments of inflammation markers from elevated Hounsfield units (HU's), chamber volumes and shapes, etc.). In some embodiments, the plurality of image based assessment features 304 that characterize pericoronary adipose tissue may include spatial distributions (e.g., artery, lengthwise, and radial ROIs), elevated HU values associated with inflammation and HU values near calcifications (e.g., including HU histograms, mean/median HU, and AHU radially), etc. In some embodiments, the plurality of image based assessment features 304 that characterize intramyocardial adipose tissue may include features that can be associated with silent myocardial infarction such as volumes, HU statistics, and location (e.g., distance to the epicardial surface). As not all fat deposits are remnants of a silent myocardial infarction, such features will help identify high-risk deposits.

The one or more of the ECG based assessment features 306 may include a duration of an interval (e.g., a PR interval, a QRS interval, a QT interval, etc.), an area of a wave (e.g., a P area), an axis of an interval (e.g., a QRS axis), an amplitude of a wave (e.g., a P wave amplitude), a heart rate of a full recording, and/or the like. In some embodiments, the feature extraction stage 116 may generate a median beat from a 10-sec 12-lead ECG, and one or more of the ECG based assessment features 306 (e.g., PR interval, QRS interval, QT interval, and P wave amplitude) may be extracted using the median beat. In some embodiments, one or more of the ECG based assessment features 306 (e.g., heart rate) may also be extracted from full ECG recordings.

The plurality of assessment features 118, including the image based assessment features 304 and the ECG based assessment features 306, are provided as input data 308 (e.g., a 1-dimensional input vector or a multi-dimensional matrix) to a machine learning stage 120 comprising one or more machine learning models. The input data 308 may have values 310 that correspond to the clinical factors 114, the image based assessment features 304, and/or the ECG based assessment features 306. The machine learning stage 120 is configured to generate a medical assessment 122 by operating upon the input data 308 to determine weightings associated with the values 310. The weightings assign different levels of importance to various features within the input data 308, thereby influencing their impact on the medical assessment 122. The image based assessment features 304 and the ECG based assessment features 306 may be collectively or separately be used to help make a decision regarding additional diagnostic procedures for the OCAD patient 102.

In some alternative embodiments, the feature extraction stage 116 and the machine learning stage 120 may be part of a deep learning stage. In such embodiments, the deep learning stage is configured to use deep learning to automatically extract features and generate a medical assessment from the data 107 (e.g., the imaging data and/or the ECG data). The deep learning stage may comprise one or more artificial neural networks (e.g., convolutional neural networks, transformers, etc.) run on one or more processors (e.g., CPUs, GPUs, and/or the like).

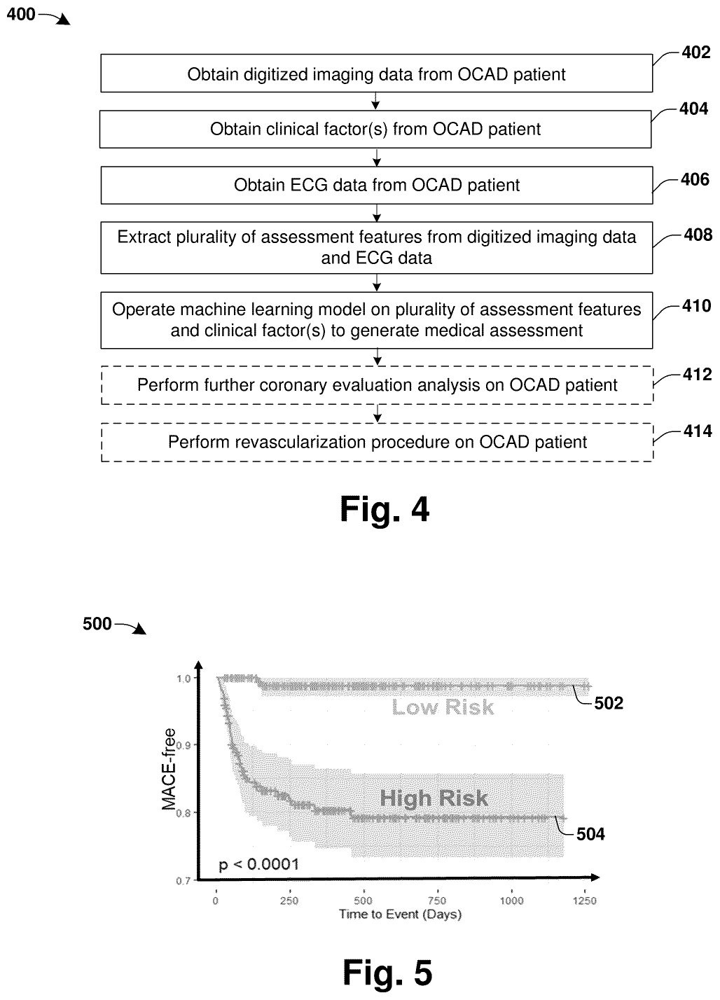

FIG. 4 illustrates a flow diagram showing some embodiments of a method 400 of operating a machine learning stage on assessment features to generate a medical assessment.

At act 402, digitized imaging data is obtained from a patient (e.g., an OCAD patient). In various embodiments, the digitized imaging data may comprise one or more digitized images and/or one or more regions of interest within a digitized image.

At act 404, one or more clinical factors are obtained from the patient.

At act 406, electrocardiogram data is obtained from the patient.

At act 408, a plurality of assessment features are extracted from the digitized imaging data and the electrocardiogram data.

At act 410, a machine learning model is operated on the plurality of assessment features and the one or more clinical factors to generate a medical assessment. In some embodiments, the medical assessment may be for additional diagnostic testing to identify obstructive coronary artery disease. In other embodiments, the medical assessment may additionally or alternatively provide for an assessment of atherosclerotic disease, disease probabilities (e.g., a probability of fat inflammation, NAFLD, etc.), a risk prediction (e.g., a risk of a coronary event within a predetermined time period of 10 years), and/or the like.

At act 412, further coronary evaluation analysis may be performed on the patient.

At act 414, a revascularization procedure may be performed on the patient.

FIG. 5 illustrates some embodiments of a graph 500 showing a likelihood of a major adverse cardiovascular event (MACE) occurring as a function of time. The graph 500 is generated by a model that uses both image based assessment features and ECG based assessment features to generate a prediction of MACE.

Graph 500 illustrates a probability of an OCAD patient being MACE free on the y-axis and a time on the x-axis. Line 502 represents a likelihood of MACE for a low risk patient, while line 504 represents a likelihood of MACE for a high risk patient. As can be seen in graph 500, a model that uses both image based assessment features and ECG based assessment features can achieve good separation between the high risk patient and the low risk patient. For example, C-indices associated with models operating upon ECG and calcium-omics may be approximately 0.84. This is significantly higher than the C-indices associated with models operating upon just calcium-omics (e.g., approximately 0.76) and models operating upon just Agatston scores (e.g., approximately 0.73), thereby showing that the use of ECG based assessment features within a model aids in a prediction of ischemia.

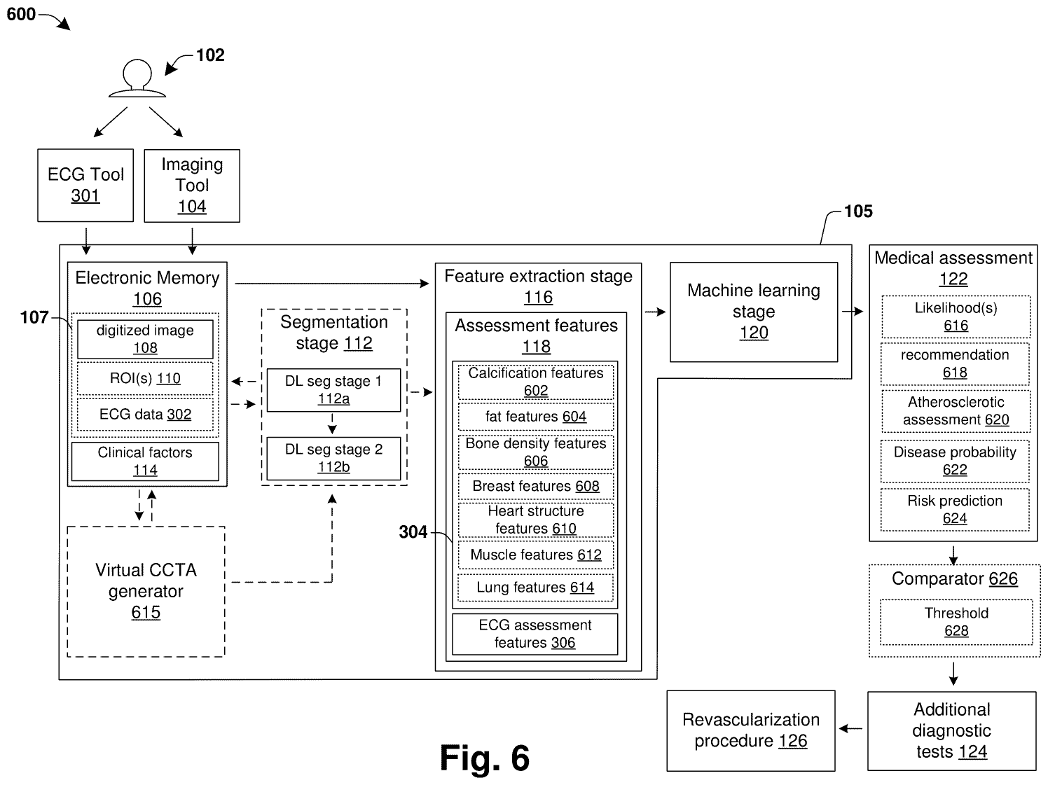

FIG. 6 illustrates some additional embodiments of a cardiovascular assessment system 600 comprising a machine learning stage configured to generate a medical assessment.

The cardiovascular assessment system 600 comprises an electronic memory 106 configured to store data 107 for an OCAD patient 102. In some embodiments, the data 107 may comprise digitized imaging data that includes one or more digitized images 108 and ECG data 302. The one or more digitized images 108 may include cardiovascular computed tomography (CT) images (e.g., CT images stored in DICOM image format), a sonogram, or the like. For example, in various embodiments the one or more digitized images 108 may comprise non-contrast, ECG (electrocardiogram) gated computed tomography (CT) images of the patient's chest and/or contrast, ECG gated CT images of the chest that have been modified for contrast. In some embodiments, the electronic memory 106 may be further configured to store one or more clinical factors 114 relating to the OCAD patient 102. In some embodiments, the electronic memory 106 may comprise a solid state memory, SRAM (static random-access memory), DRAM (dynamic random-access memory), and/or the like.

In some embodiments, a segmentation stage 112 is in communication with the electronic memory 106. The segmentation stage 112 is configured to segment the one or more digitized images 108 (e.g., non-contrast digitized images) to generate one or more segmented digitized images that respectively identify one or more regions of interest (ROI) 110 (e.g., volumes of interest). In some embodiments, the one or more ROI 110 may comprise a heart, calcifications (e.g., coronary calcification, aortic calcifications, aortic valve calcifications, mitral annulus calcifications, or the like), fat tissue (e.g., epicardial fat, liver fat, pericardial fat, subcutaneous fat, periaortic fat, pericoronary fat, and/or the like), heart structures (e.g., whole heart, right atrium, right ventricle, left atrium, left ventricle, aortic root, etc.), bone (e.g., spin thoracic vertebrae, etc.), muscle (e.g., skeletal muscle, pectoralis muscle, etc.), a lung, breast tissue, a liver, a spine thoracic vertebrae, and/or the like. In some embodiments, the one or more segmented digitized images may comprise and/or be one or more binary masks. In some such embodiments, the one or more binary masks comprise images having a value of “1” in image units (e.g., pixels, voxels, etc.) identified as being within the one or more ROI 110 and having a value of “0” in image units outside of the one or more ROI 110.

In some embodiments, the segmentation stage 112 may comprise one or more deep learning models (e.g., artificial neural networks). For example, the segmentation stage 112 may comprise a graphical neural network (GNN) that utilizes an adaptive Hounsfield Unit (HU)-attention-window. In some embodiments, the segmentation stage 112 may utilize transformer models, which use a self-attention mechanism to differentially weigh a significance of each input data element. The self-attention mechanism may improve a GNN's ability to formulate geometric and semantic relationships between the one or more ROIs 110 (e.g., volumes of interest). In some embodiments, the one or more deep learning models may be run on one or more processors (e.g., a central processing unit including one or more transistor devices configured to operate computer code to achieve a result, a microcontroller, a graphics processing unit, and/or the like).

In some embodiments, the segmentation stage 112 may utilize a two-step stage deep learning segmentation approach. In such embodiments, a first deep learning segmentation stage 112a may be configured to process a digitized image at a lower resolution to identify larger regions of interest (e.g., a heart and aorta, a liver, a spine thoracic vertebrae including back musculature, etc.). A second deep learning segmentation stage 112b is subsequently configured to use deep learning semantic segmentation to identify actual tissues of interest. For example, a heart and aorta volume identified by the first deep learning segmentation stage 112a may be further processed by the second deep learning segmentation stage 112b to identify the cardiac chambers, epicardial fat, pericardial fat, and calcifications in the coronaries, aorta, and valves. In another example, the first deep learning segmentation stage 112a may be used to segment a heart from surrounding fat tissue, and the second deep learning segmentation stage 112b may be used to segment pericardial and epicardial adipose tissue from the surrounding fat tissue.

It has been appreciated that it may be helpful to take into consideration the locations of vessels within a digitized image during segmentation of pericoronary adipose tissue. Therefore, in some embodiments the segmentation stage 112 may be configured to identify locations of vessels within a digitized image prior to performing segmentation to identify pericoronary adipose tissue. For example, vessel center lines may be identified from a vessel and then progressively dilated in 3D to form a new shell around the centerline with each dilation. Within each shell, thresholds (e.g., HU between −190 and −30 HU) may be applied to segment potential pericoronary adipose tissue. Dilation may stop at a predetermined value beyond which pericoronary adipose tissue is not thought to exist. The results will be a centerline and encapsulating pericoronary adipose tissue.

In some embodiments, the vessels may be identified using a semiautomated segmentation of vessels. In some such embodiments, an image volume may be processed with a vesselness filter to enhance blood vessels in 3D. In some embodiments, a user may specify a start and end of the vessel segment, and the segmentation stage 112 will identify the best vessel segment center-line connecting these points using a graphical optimization algorithm (e.g., using 3D dynamic programming). In other embodiments, a user may initiate a seed voxel in a vessel segment and then the segmentation stage 112 will run dynamic programming to give a cost to each voxel in the image. The user then interactively identifies potential end voxels.

In other embodiments, the vessels may be identified using an automated segmentation of vessels. In some such embodiments, a deep learning approach may be used within a heart volume of interest. For example, a fully convolutional network (FCN) configured to receive an original image volume and a processed volume (where noise has been reduced and vessels have been enhanced with 3D vesselness) may be trained with CT calcium score images and vessel labels. Vessel labels will be obtained from corresponding CTA images which have been registered to a corresponding CT calcium score image volume to generate a ground truth for deep learning. The output of this semantic segmentation method will be a probability at each voxel of being a coronary artery voxel. A threshold applied to this volume will give vessels and eliminate noise. In some embodiments, connected components having a number of voxels less than a number for consideration may be eliminated. In some embodiments, a 3D thinning operation may be used to get a vessel centerline.

In some embodiments, a virtual CCTA image generator 615 may be configured to generate a virtual CCTA image from a CTCS image using a deep learning, image translation approach. The virtual CCTA image may be saved as part of the data 107 within the electronic memory 106. The virtual CCTA image may be subsequently operated upon by the segmentation stage 112 to identify the one or more ROI 110. In some embodiments, the virtual CCTA image generator 615 may comprise a general adversarial network (GAN). The virtual CCTA image generator 615 may be run on one or more processors (e.g., a central processing unit including one or more transistor devices configured to operate computer code to achieve a result, a graphics processing unit (GPU), a microcontroller, and/or the like).

A feature extraction stage 116 is configured to extract a plurality of assessment features 118 from the one or more ROI 110. The plurality of assessment features 118 may include features that are prognostic of obstructive coronary artery disease and/or ischemia. The plurality of assessment features 118 may include image based assessment features 304 and ECG based assessment features 306. In some embodiments, the plurality of assessment features 118 may include hand-crafted features that have been found to be related to cardiovascular disease, obstructive coronary artery disease, metabolic disease, ischemia, and/or the like. In other embodiments, the plurality of assessment features 118 may comprise deep learning features. In yet other embodiments, the plurality of assessment features 118 may comprise a combination of hand-crafted features and deep learning features.

In various embodiments, the plurality of assessment features 118 may include one or more of calcification features 602, fat features 604, bone density features 606, breast features 608, heart structure features 610, muscle features 612, lung features 614, and/or the like. In some embodiments, the feature extraction stage 116 may be implemented as computer code run by a processing unit (e.g., a central processing unit including one or more transistor devices configured to operate computer code to achieve a result, a microcontroller, or the like).

In some embodiments, the calcification features 602 may comprise structural features, a whole heart Agatston, a calcification mass score, a whole heart calcification mass, an aortic calcification mass, and/or the like. The calcification features 602 may characterize calcifications in coronary arteries, extra-coronary calcifications in an aorta, an aortic valve, and/or a mitral annulus. The calcifications in the coronary arteries indicate that there is coronary artery disease, as calcifications are not found in young, healthy arteries. The presence of calcifications in the aortic valve can also lead to future failure of the valve. Therefore, a presence of calcifications indicates the presence of vascular disease with implications for coronary artery disease.

In some embodiments, the calcification features 602 may be extracted using a virtual CCTA image. In such embodiments, a virtual CCTA image may be segmented to identify coronary arteries, thereby improving coronary calcification identification and attribution of a calcification to a particular coronary artery. For example, candidate calcifications may be obtained using a standard rule (e.g., ≥130-HU & 3-connected) within a pericardial sac. A 3D patch centered may be extracted from each candidate calcification that is sufficiently large to capture the anatomy and apply deep learning classification to determine coronary calcifications and their coronary artery territory membership. Information on the nearness of coronaries may be provided from a virtual CCTA image.

In some embodiments, the fat features 604 may comprise features extracted from one or more of liver fat, epicardial fat, pericardial fat, subcutaneous fat, periaortic fat, and pericoronary fat. The liver fat features may give an indication of metabolic syndrome and/or fibrosis and can be assessed by determining fat concentration in the liver from CT intensity values (e.g., mean intensity values), morphometrics (e.g., shape, texture, etc.), and/or other CT intensity features (e.g., a standard deviation, kurtosis, histogram, etc.). Epicardial fat features may include features from visceral fat between the pericardium and the epicardial surface of the heart, which can be assessed using morphometrics (e.g., volume and shape), CT intensity values, and/or textures. Epicardial fat features may give an indication of pro-inflammatory mediators that can worsen endothelial dysfunction and lead to coronary artery disease. Pericardial fat features may include features from visceral fat along the external surface of the pericardium, which can be assessed using morphometrics (e.g., volume and shape), CT intensity values, and/or textures. Pericardial fat features may be associated with cardiovascular disease and an increased risk of blood glucose level, systolic blood pressure, hypercholesterolemia, and/or atrial fibrillation. Subcutaneous fat features may include features from subcutaneous fat just under the skin, which can be assessed using morphometrics (e.g., volume and shape), CT intensity values, and/or textures. Subcutaneous fat features may be associated with metabolic syndrome and/or cardiovascular disease. Periaortic fat features may include features from fat surrounding the aorta, which can be assessed using morphometrics (e.g., volume and shape), CT intensity values, and/or textures. Periaortic fat features may be associated with cardiovascular disease, water content, and/or inflammation. Pericoronary fat features may include features from fat surrounding the coronary arteries, which can be assessed using morphometrics (e.g., volume and shape), CT intensity values, and/or textures. Pericoronary fat features may be associated with major adverse cardiovascular events.

In some embodiments, the fat features 604 may comprise thickness measurements corresponding to a “layer” of fat identified in a segmentation process. For example, a layer of fat may have a plurality of thicknesses determined between a plurality of points on an inner surface and a plurality of points on an outer surface. A set of thickness values for the layer of fat may be determined from standard statistical measures (e.g., a mean, standard deviation, minimum, maximum, kurtosis, and histogram) of the plurality of thicknesses. Fat features (e.g., pericoronary fat features) may include features based upon intensity values (e.g., mean, standard deviation, kurtosis, and/or histogram) and/or intensity gradients from a center line (e.g., mean, standard deviation, kurtosis, and/or histogram). In some embodiments, one or more fat features 604 may be extracted from a virtual CCTA image.

In some embodiments, the bone density features 606 may be assessed from CT intensity values in spine thoracic vertebrae and may include bone mineral density, morphometrics (e.g., volume and shape), and/or intensity textures. The bone density features 606 may be a biomarker of calcium metabolism and/or cardiovascular disease.

In some embodiments, the breast features 608 include features that characterize breast arterial calcifications, due to their association with cardiovascular risk. The breast features 608 may include breast tissue morphometrics, texture, and/or a presence and/or amount of calcification.

In some embodiments, the heart structure features 610 may include morphometrics (e.g., volume and shape) and/or intensity features. The heart structure features 610 characterize heart structures such as whole heart, right atrium, right ventricle, left atrium, left ventricle, and/or aortic root. The heart structure features 610 may be indicative of future heart failure. In some embodiments, the heart structure features 610 may include an overall heart size, a left ventricular hypertrophy, a left atrium/right atrium size, intensity textures, and/or the like. In some embodiments, one or more of the heart structure features 610 may be extracted from a virtual CCTA image.

In some embodiments, the muscle features 612 may include CT intensity values, morphometrics (e.g., volume and shape), and/or intensity textures. The muscle features may assess skeletal muscle (e.g., pectoralis muscle) for indications of muscle loss (e.g., sarcopenia) and/or frailty. The muscle features 612 may be indicative of mortality in patients with cardiovascular disease.

In some embodiments, the lung features 614 may include features that analyze lung size, texture, and/or presence of nodules as a marker of cardiovascular disease. The lung features 614 may also include features that analyze artery shape and size as a marker of pulmonary hypertension.

A machine learning stage 120 is configured to utilize the plurality of assessment features 118 to generate a medical assessment 122 (e.g., for additional diagnostic testing to identify obstructive coronary artery disease and/or ischemia). In some embodiments, the medical assessment 122 may comprise a numeric score. In some embodiments, the numeric score may comprise a numeric value and/or percentage that indicates a likelihood 616 that additional diagnostic testing would be beneficial to the OCAD patient 102.

For example, the medical assessment 122 may comprise a likelihood that a CCTA would yield a binary determination of positive obstructive disease as determined from visual inspection via a reporting system such as Coronary Artery Disease Reporting and Data System (CAD-RADS) or one of its variations, a likelihood that a CCTA would yield a binary determination of positive obstructive disease as determined from visual inspection via a reporting system such as CAD-RADS or one of its variations, or fractional flow reserve (FFR) as obtained from a computational evaluation of the CCTA (e.g., CTFFR from HeartFlow), a likelihood that a CCTA would determine marginal or more severe obstructive disease as determined from visual/software-assisted inspection via a reporting system such as CAD-RADS or one of its variations or from a flow wire measurement, a likelihood that a CCTA would determine marginal or more severe obstructive disease as determined from visual/software-assisted inspection via a reporting system such as CAD-RADS or one of its variations, or FFR as obtained from a computational evaluation of the CCTA (e.g., CTFFR from HeartFlow) or from a flow wire measurement, a likelihood of any other assessment that would suggest any degree of obstructive disease, and/or a likelihood of any other non-obstructive assessment of disease from CCTA. In some embodiments, the likelihood 616 may comprise a weighted mix of the above likelihoods.

In other embodiments, the medical assessment 122 may comprise a likelihood that an additional diagnostic test would be ordered based on historical data from an institution (e.g., a hospital) and/or a geographical region (e.g., the United States). In such embodiments, a physician could determine whether he or she is making a diagnosis that is within the norms of an institution and/or geographical region. In some such embodiments, the medical assessment 122 may comprise a likelihood that CCTA would be ordered based on historical data, a likelihood that CCTA or x-ray angiography would be ordered based on historical data, and/or a likelihood that CCTA, x-ray angiography, or further diagnostic test (e.g., one of the multiple kinds of stress tests) would be ordered based on historical data.

In yet other embodiments, the medical assessment 122 may comprise a recommendation 618 to a health care professional. For example, the recommendation 618 may include a recommendation for a CCTA, for active surveillance, or to do nothing. In some embodiments, the recommendation 618 may be generated by applying one or more thresholds 628 to a numeric score (e.g., to the likelihood 616). For example, a comparator 626 may comprise a value of a likelihood 616 to one or more threshold values 628 (e.g., of 50%, 80%, or the like) to generate a recommendation for a CCTA. In some embodiments, the one or more threshold values 628 may have a value that is less than 50% (e.g., that is equal to approximately 20%) to limit numbers of false-negatives at the expense of more false-positives.

In yet other embodiments, the medical assessment 122 may comprise an assessment of atherosclerotic disease 620, disease probabilities 622 (e.g., a probability of fat inflammation, NAFLD, etc.), a risk prediction 624 (e.g., a risk of a coronary event within a predetermined time period of 10 years), and/or the like.

In some embodiments, the machine learning stage 120 may comprise one or more machine learning models, such as one or more of a regression model, a Cox Hazard regression model, a support vector machine (SVM), a linear discriminant analysis (LDA) classifier, a Naïve Bayes classifier, a Random Forest, Adaboost, a convolutional neural network, a transformer model, and the like. In some embodiments, the machine learning stage 120 may be run on one or more processors (e.g., a central processing unit including one or more transistor devices configured to operate computer code to achieve a result, a graphics processing unit (GPU), a microcontroller, or the like).

By using the plurality of assessment features 118 to generate the medical assessment 122, an informed and accurate decision can be made regarding the benefit of performing additional diagnostic tests on the OCAD patient 102. By making an informed and accurate decision regarding additional diagnostic tests, unnecessary testing can be avoided thereby better controlling the cost of treatment for the OCAD patient 102, risks for the OCAD patient 102 (e.g., due to radiation exposure), and/or resources for a health care provider.

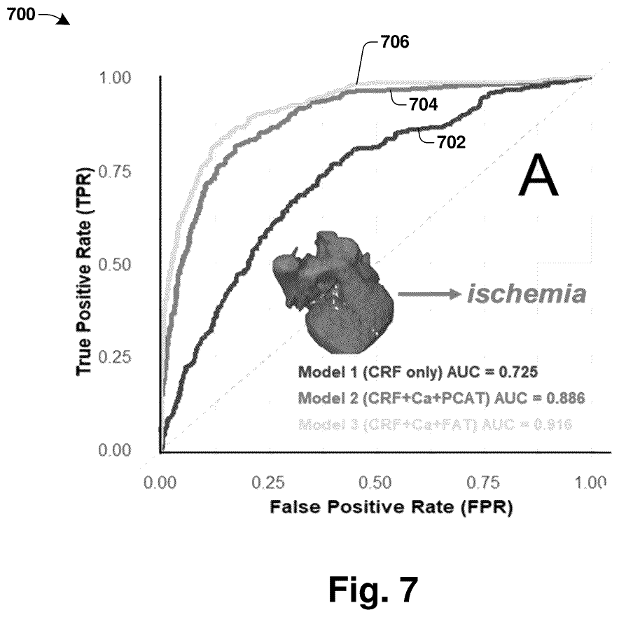

FIG. 7 illustrates an example receiver operating characteristic (ROC) curve 700 corresponding to a risk of ischemia. The ROC curve 700 plots a true positive rate on a y-axis and a false positive rate on the x-axis.

ROC curve 700 includes a first line 702 corresponding to a first model trained on clinical risk factors. The ROC curve 700 also includes a second line 704 corresponding to a second model trained on clinical factors, calcification features, and pericoronary adipose tissue (PCAT) features. The ROC curve 700 also includes a third line 706 corresponding to a third model trained on clinical factors, calcification features, and fat-features (e.g., including PCAT features and adipose fat features). As can be seen in ROC curve 700, the third model achieves a highest AUC (e.g., an AUC of approximately 0.92) for prediction of ischemic disease. Therefore, the combination of clinical factors, calcification features, and fat-features has a high predictive ability for identifying ischemic disease.

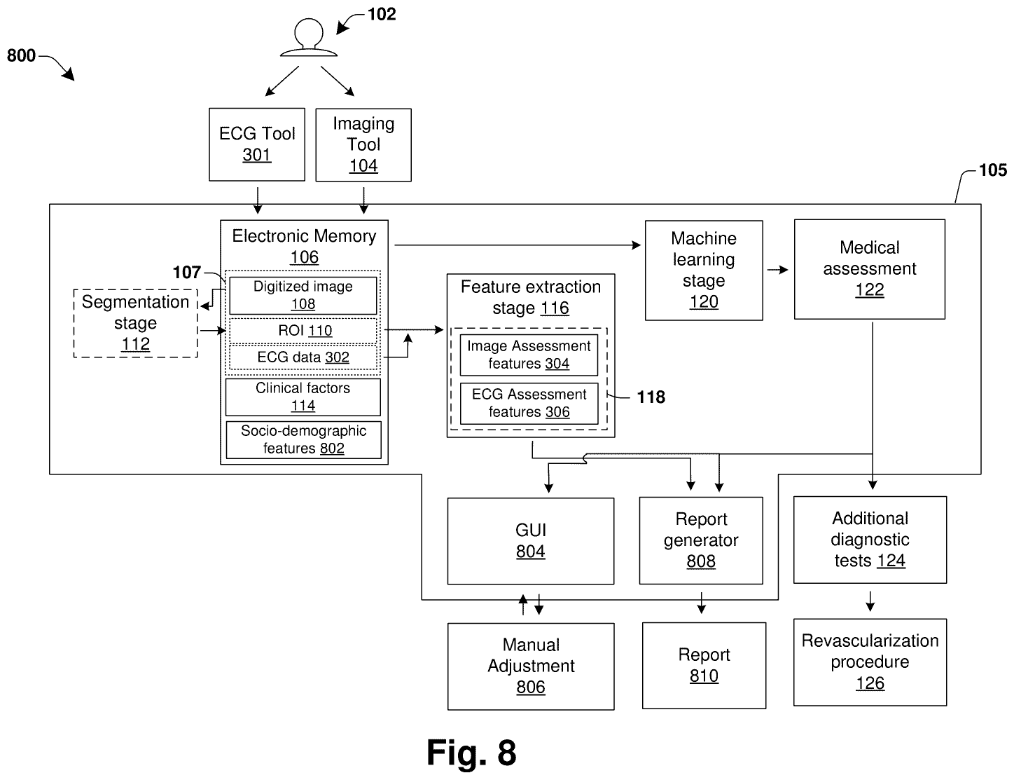

FIG. 8 illustrates some additional embodiments of a cardiovascular assessment system 800 comprising a machine learning stage configured to generate a medical assessment.

The cardiovascular assessment system 800 comprises a cardiovascular assessment apparatus 105 that includes an electronic memory 101 configured to store data 107 relating to an OCAD patient 102. In some embodiments, the data 107 may comprise one or more digitized images 108 (e.g., CTCS images), one or more regions of interest (ROI) 110, and ECG data 302. In additional embodiments, the electronic memory 101 is configured to store one or more clinical factors 114 (e.g., a blood pressure, chest pain, lipid assessments, presence of comorbidities such as diabetes and psoriasis high cholesterol, obesity, a body mass index (BMI), and/or the like). In yet additional embodiments, the electronic memory 101 is configured to store one or more socio-demographic factors 802, such as a race, an age, a biological sex, an income, an education, an occupation, and/or the like.

A feature extraction stage 116 is configured to extract a plurality of assessment features 118 from the data 107. The plurality of assessment features 118 include features that have been found to be prognostic of obstructive coronary artery disease atherosclerotic events, and/or ischemia. The plurality of assessment features may include image based assessment features 304 extracted from the one or more ROI 110 and ECG based assessment features 306 extracted from the ECG data 302.

A machine learning stage 120 is configured to utilize the plurality of assessment features 118, the one or more clinical factors 114, and the one or more socio-demographic factors 802 to generate a medical assessment 122. The medical assessment 122 may indicate whether or not the OCAD patient 102 would benefit from additional diagnostic tests to identify the presence and extent of obstructive coronary artery disease.

In some embodiments, the cardiovascular assessment apparatus 105 may further comprise a graphic user interface (GUI) 804. The GUI 804 may be configured to display the one or more segmented digitized images and/or the medical assessment 122 associated with the OCAD patient 102. The GUI 804 is configured to allow a user to manually identify the ROI 110 within one or more segmented digitized images and/or to edit the medical assessment 122. In some embodiments, the GUI 804 may be configured to allow a user to review segmented images and make manual adjustments 806 to edit the segmented images. Editing the segmented images may allow a user to better identify a ROI. In some embodiments, edits from the user may be fed back to the segmentation stage 112 to improve segmentation.

In some embodiments, the cardiovascular assessment apparatus 105 may also include a report generator 808. The report generator 808 is configured to generate a report 810 based upon the assessment features 118 and/or the medical assessment 122. The report 810 describes variables that led to a determination for additional diagnostic tests (e.g., numeric measurements of whole-heart Agatston, liver fat, mineral bone density, volume of pericardial fat, etc.), a disease probability (e.g., a probability of fat inflammation, probability of NAFLD), a risk prediction of a major adverse cardiovascular event, an assessment of atherosclerotic disease, and/or the like. In some additional embodiments, the report 810 may include an easy-to-understand description of topics like cardiovascular risk probabilities and the advantages of CCTA. In some additional embodiments, the report 810 may include assessments generated by separate models for ECG, CTCS, and clinical features, and then combining results to see the relative contribution of input feature groups. This might be important for explaining results to physicians and patients. The report 810 may be shared with a health care professional to further improve diagnosis of the OCAD patient 102 and/or decision making for the health care professional and/or the OCAD patient 102. The report 810 may improve patient education. It has been appreciated that such patient education may improve patient adherence to a prescribed drug (e.g., a statin to reduce lipids) and/or lifestyle changes (e.g., smoking cessation, weight loss, and exercise).

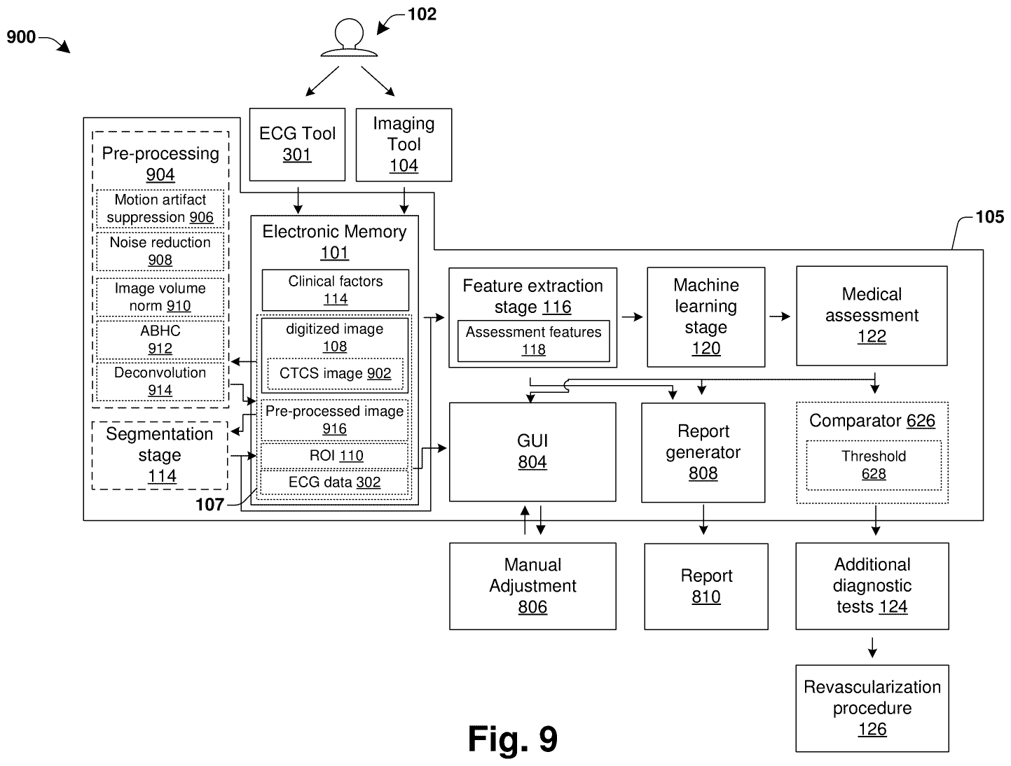

FIG. 9 illustrates some additional embodiments of a cardiovascular assessment system 900 comprising a machine learning stage configured to generate a medical assessment.

The cardiovascular assessment system 900 comprises a cardiovascular assessment apparatus 109 that includes an electronic memory 101 configured to store data 107 relating to an OCAD patient 102. In some embodiments, the data 107 may comprise one or more digitized images 108 (e.g., CTCS images), one or more ROI 110, and ECG data 302. The electronic memory 106 may also be configured to store one or more clinical factors 114.

In some embodiments, the cardiovascular assessment apparatus 109 may further comprise a pre-processing pipeline 904. The pre-processing pipeline 904 is configured to perform one or more pre-processing operations 906-914 upon the one or more digitized images 108 to enhance the one or more digitized images 108 and to generate one or more pre-processed images 916. In various embodiments, the one or more pre-processing operations 906-914 include motion artifact suppression 906, noise reduction 908, image volume normalization 910, automated beam hardening correction (ABHC) 912, and/or deconvolution 914 (e.g., for coronary and extra-coronary calcifications).

In some embodiments, motion artifact suppression 906 may utilize deep learning to improve data quality by minimizing a cost function related to calcium motion. To train the deep learning network, a paired data set including moving and static images is generated by using a CT simulator to move calcium with a defined direction and velocity. The deep learning network uses moving images as input and generates output images with a minimized error to static images.

In some embodiments, noise reduction 908 may utilize a general adversarial network (GAN) to reduce noise due to low dose acquisition. The GAN may utilize an algorithm that has been trained on a paired data set including low-dose and high-dose images which are generated from a CT simulator, a physical phantom, a cadaver heart, and/or clinical data with low and high acquisitions.

In some embodiments, image volume normalization 910 may utilize a machine learning model to make images look similar with regard to slice thickness and noise. Image volume normalization attempts to normalize data obtained from different CT scanners and with different acquisition parameters (e.g., different dose levels and slice thicknesses) to improve quantification. To generate normalized volume, training data may be used. The training data may consist of CT volumes with different acquisition parameters (e.g., dose and slice thickness) and a reference volume. In some embodiments, image volume normalization may be performed using a general adversarial network (GAN), a CycleGAN, or other CNN networks.

In some embodiments, automated beam hardening correction 912 may be configured to reduce beam hardening artifacts which can otherwise reduce Hounsfield unit (HU) values (e.g., thereby being interpreted as low attenuated material) and which may interfere with accurate and precise quantitative calcium score and fat analysis. The image-based ABHC algorithm automatically determines correction parameters for a beam hardening correction model and applies them to reduce artifacts in the image.

In some embodiments, deconvolution 914 may be applied to patient CT volumes to get more accurate measurement of coronary and extra-coronary calcifications. Deconvolution may be performed by assuming the CT system is linear and spatially invariant, so that the output blurred image with additive noise is given by:

$$I ( x , y , z ) = \int \int \int f ( {x}^{′} , {y}^{′} , {z}^{′} ) h ( x - {x}^{′} , y - {y}^{′} , z - {z}^{′} ) {dx}^{′} {dy}^{′} {dz}^{′} + n ( x , y , z ) = f ( x , y , z ) * h ( x , y , z ) + n ( x , y , z )$$

where I(x, y, z) is the measured image volume from the CT system, f(x, y, z) is the idealized input image, ‘*’ denotes convolution, h(x, y, z) represents the 3D PSF, and n is additive noise. In some embodiments, to estimate f(x, y, z), the method maximizes the likelihood of obtaining the output image data assuming Poisson noise statistics. The log likelihood is maximized in an iterative fashion. The PSF is assumed to be known, and the method constrains f to be non-zero. To reduce the effects of noise amplification, the method is modified to reduce noise. A damping coefficient is applied as estimated from the noise in CT images. To reduce the effects of noise even further, we use an anisotropic diffusion filter. In other embodiments, different types of convolution may be used, such as a blind deconvolution

The one or more pre-processed images 916 are provided to a segmentation stage 112. The segmentation stage 112 is configured to segment the one or more pre-processed images 916 to generate one or more segmented digitized images (e.g., label volumes) that identify regions of interest such as a lung, a heart, fat tissue, etc.

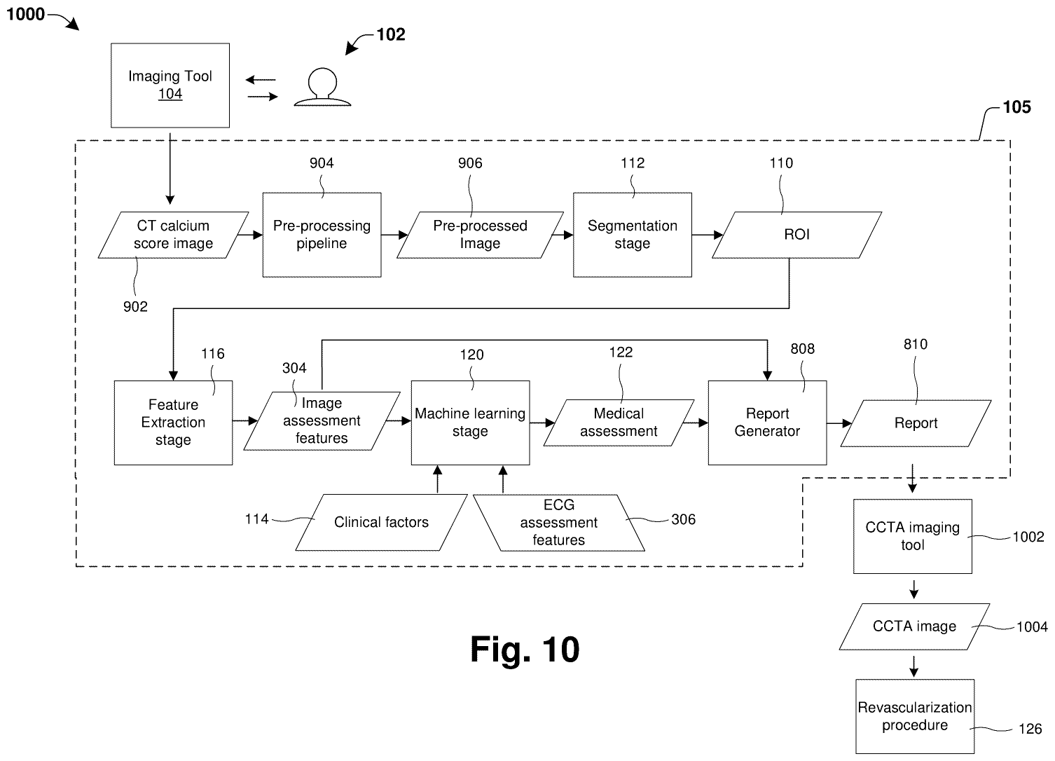

FIG. 10 illustrates some embodiments of a block diagram 1000 showing an exemplary operation of a disclosed cardiovascular assessment apparatus 105 on a computer tomography calcium score (CTCS) image.

As shown in block diagram 1000, a cardiovascular assessment apparatus 105 is configured to receive a CT calcium score (CTCS) image 902 from an imaging tool 104 operated upon an OCAD patient 102. In some embodiments, the CTCS image 902 may comprise a CT volume with 512×512 voxels in an x-y plane and about 70 slices in a z dimension with 0.7×0.7×2.5 mm resolution. In other embodiments, the CTCS image 902 may comprise other volume sizes, numbers of slices, and/or resolutions.

A pre-processing pipeline 904 is configured to perform pre-processing operations on the CTCS image 902 to form a pre-processed image 916.

A segmentation stage 112 is configured to operate upon the pre-processed image 916 to generate one or more segmented digitized images that include label volumes identifying regions of interest (ROI) 110. The segmentation stage 112 may comprise a 3D U-NET with generalized Dice score as a loss function. In some embodiments, the segmentation stage 112 may generate bounding box VOIs by cropping a full size CT volume to bounding boxes surrounding segmentations.

A feature extraction stage 116 is configured to extract a plurality of image based assessment features 304 from the ROI 110 within the one or more segmented digitized images. The plurality of image based assessment features 304 are provided to a machine learning stage 120, which utilizes the plurality of image based assessment features 304, along with one or more clinical factors 114 and a plurality of ECG based assessment features 306, to generate a medical assessment 122 corresponding to a benefit of additional diagnostic tests.

In some embodiments, after the medical assessment 122 is generated, the medical assessment 122, the plurality of image based assessment features 304, the one or more clinical factors 114, and the plurality of ECG based assessment features 306 may be provided to a report generator 808 that is configured to generate a report 810 that can be provided to a health care professional. The report 810 may include the medical assessment 122 and/or disease probabilities. The report 810 may, for example, be laid out in such a way that a lay-person (e.g., a non-clinician) can easily understand the report 810.

In some embodiments, based upon the medical assessment 122, a CCTA imaging tool 1002 may be utilized to generate a CCTA image 1004 of the OCAD patient 102. The CCTA image 1004 may be used by a health care professional to make a determination as to whether the OCAD patient 102 would benefit from a revascularization procedure 126 (e.g., a stent, angioplasty, bypass surgery, and/or the like).

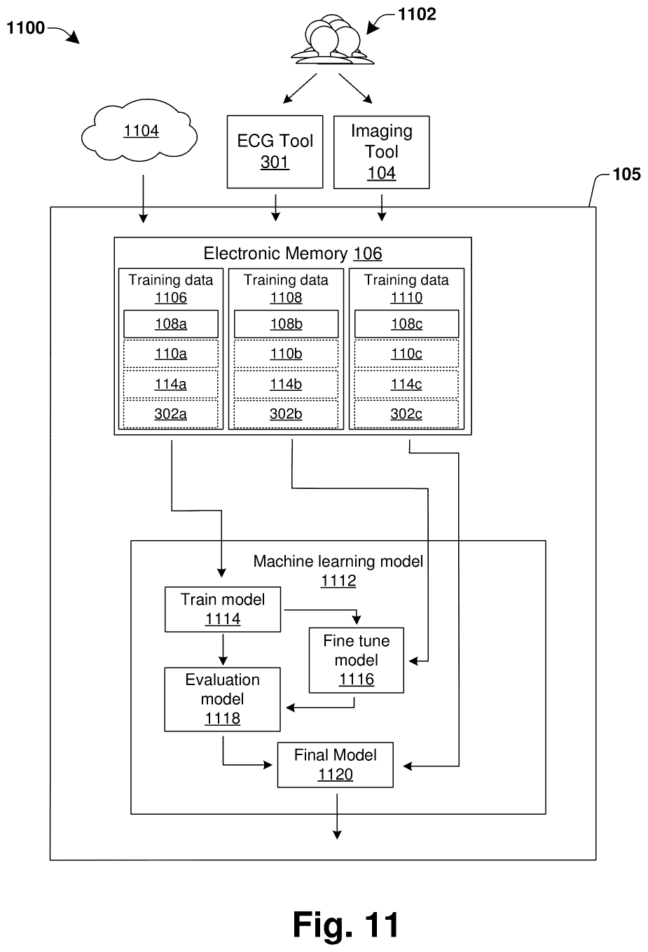

FIG. 11 illustrates some additional embodiments of a cardiovascular assessment system 1100 comprising a machine learning stage configured to generate a medical assessment.

The cardiovascular assessment system 1100 comprises an electronic memory 106 configured to store data relating to a plurality of OCAD patients 1102. The electronic memory 106 is configured to store the data as a plurality of different data sets 1106-1110. In some embodiments, the plurality of different data sets 1106-1110 may respectively comprise one or more clinical factors, one or more digitized images, one or more segmented images, and/or ECG data. The plurality of different data sets 1106-1110 may include a training data set 1106, a testing data set 1108, and/or a validation data set 1110. The data may be received from an imaging tool (e.g., a CT scanner) operated upon one or more of the plurality of OCAD patients 1102, from an ECG tool 301 operated upon one or more of the plurality of OCAD patients 1102, and/or downloaded from an online database 1104 (e.g., an online archive).

The training data set 1106, the testing data set 1108, and/or the validation data set 1110 may respectively comprise digitized images 108a-108c, segmented digitized images 110a-110c, one or more clinical factors 114a-114c, and ECG data 302a-302c relating to a subset of the plurality of OCAD patients 1102. In some embodiments, the data stored in the electronic memory 106 may be split into the training data set 1106, the testing data set 1108, and the validation data set 1110 at a ratio of 80%/10%/10%.

The training data set 1106, the testing data set 1108, and/or the validation data set 1110 may be used to train and validate one or more downstream machine learning models 1112. In various embodiments, the one or more downstream machine learning models 1112 may include one or more of a pre-processing pipeline (e.g., including GAN models), a segmentation stage (e.g., including deep learning segmentation models), a feature extraction stage, and/or a machine learning stage. The training data set 1106 may be used to train initial versions 1114 of the one or more downstream machine learning models 1112. The initial versions 1114 of the one or more downstream machine learning models 1112 may be subsequently fine-tuned 1116 using the testing data set 1108 to generate one or more evaluation models 1118. The validation data set 1110 may then be used to generate a final version 1120 of the one or more downstream machine learning models 1112 from the one or more evaluation models 1118.

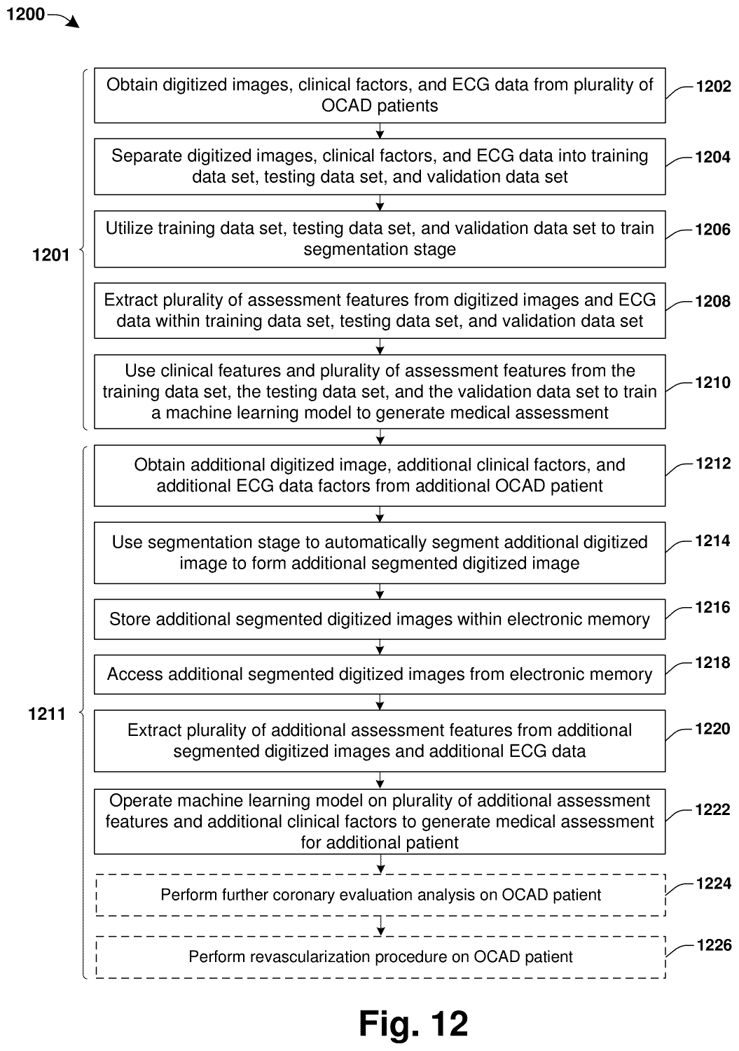

FIG. 12 illustrates a flow diagram showing some additional embodiments of a method 1200 of operating a machine learning stage on assessment features to generate a medical assessment.

The method 1200 comprises a training phase 1201 and an application phase 1211. The training phase 1201 is configured to train one or more machine learning models to generate a medical assessment (e.g., corresponding to additional diagnostic testing for obstructive coronary artery disease). In some embodiments, the training phase 1201 may be performed according to acts 1202-1210.

At act 1202, a plurality of digitized images, clinical factors, and ECG data are obtained from a plurality of obstructive OCAD patients. In some embodiments, the plurality of digitized images may comprise a plurality of non-contrast low-dose computed tomography calcium score (CTCS) images. In some embodiments, the one or more clinical factors may include a blood pressure, chest pain, lipid assessments, presence of comorbidities such as diabetes and psoriasis high cholesterol, obesity, a body mass index (BMI), and/or the like.

At act 1204, the plurality of digitized images, clinical factors, and ECG data are separated into a training data set, a testing data set, and a validation data set.

At act 1206, the training data set, the testing data set, and the validation data set are used to train a segmentation tool to automatically segment digitized images and form segmented images that identify one or more regions of interest (ROIs).

At act 1208, a plurality of assessment features are extracted from the digitized images and ECG data within the training data set, the testing data set, and the validation data set.

At act 1210, the clinical features and the plurality of assessment features extracted from the training data set, the testing data set, and the validation data set are used to train a machine learning model to generate a medical assessment (e.g., whether or not an OCAD patient would benefit from additional diagnostic tests to identify a presence and extent of obstructive coronary artery disease).

The application phase 1211 is configured to utilize the one or more trained machine learning models to generate a medical assessment for an additional OCAD patient. In some embodiments, the application phase 1211 may be performed according to acts 1212-1222.

At act 1212, additional digitized images, additional clinical factors, and additional ECG data are obtained from an additional CAD patient.

At act 1214, the additional digitized images are automatically segmented to form one or more additional segmented digitized images that identify one or more additional regions of interest (ROIs).

At act 1216, the one or more additional segmented digitized images are stored within electronic memory.

At act 1218, the one or more additional segmented digitized images are accessed from the electronic memory.

At act 1220, a plurality of additional assessment features are extracted from the one or more additional segmented digitized images and the ECG data.

At act 1222, a machine learning model is operated on the plurality of additional assessment features and the one or more additional clinical factors to generate a medical assessment (e.g., that corresponds to whether or not the additional OCAD patient would benefit from additional diagnostic tests to identify a presence and extent of obstructive coronary artery disease).

At act 1224, further coronary evaluation analysis may be performed on the OCAD patient. The further coronary evaluation analysis may include anatomic testing (e.g., CCTA, x-ray angiography, etc.) and/or functional testing (e.g., stress ECG, pharmacological or exercise SPECT and PET, pharmacological stress MRI, etc.).

At act 1226, a revascularization procedure may be performed on the OCAD patient. The revascularization procedure may include placement of a stent, a bypass surgery, an endarterectomy, and/or the like.

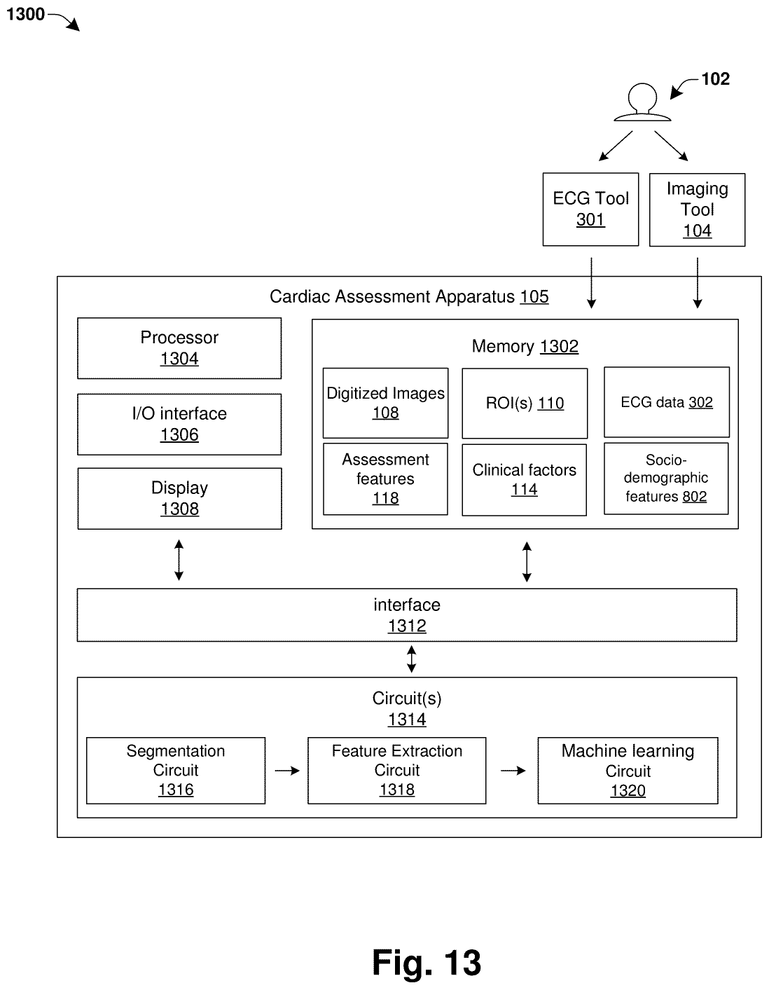

FIG. 13 illustrates a block diagram of some embodiments of a cardiovascular assessment system 1300 comprising a machine learning stage configured to generate a medical assessment.

The cardiovascular assessment system 1300 comprises a cardiovascular assessment apparatus 105. The cardiovascular assessment apparatus 105 is coupled to an imaging tool 104 (e.g., a CT imaging tool) that is configured to generate one or more digitized images 108 (e.g., CTCS images) of an OCAD patient 102. In some embodiments, the imaging tool 104 may comprise a low-dose CT scanner. The cardiovascular assessment apparatus 105 may be further coupled to an ECG tool 301 that is configured to generate ECG data 302 of the OCAD patient 102.

The cardiovascular assessment apparatus 105 comprises a processor 1304 and a memory 1302. The processor 1304 can, in various embodiments, comprise circuitry such as, but not limited to, one or more single-core or multi-core processors. The processor 1304 can include any combination of general-purpose processors and dedicated processors (e.g., graphics processors, application processors, etc.). The processor(s) 1304 can be coupled with and/or can comprise memory (e.g., memory 1302) or storage and can be configured to execute instructions stored in the memory 1302 or storage to enable various apparatus, applications, or operating systems to perform operations and/or methods discussed herein.

The memory 1302 can be further configured to store digitized imaging data comprising the one or more digitized images 108 (e.g., non-contrast digitized images) obtained by the imaging tool 104, ECG data 302 obtained by the ECG tool 301, one or more clinical factors 114 relating to the OCAD patient 102, and/or socio-demographic features 802 relating to the OCAD patient 102. The one or more digitized images 108 may comprise a plurality of pixels, each pixel having an associated intensity.

The cardiovascular assessment apparatus 105 also comprises an input/output (I/O) interface 1306 (e.g., associated with one or more I/O devices), a display 1308, one or more circuits 1314, and an interface 1312 that connects the processor 1304, the memory 1302, the I/O interface 1306, the display 1308, and the one or more circuits 1314. The I/O interface 1306 can be configured to transfer data between the memory 1302, the processor 1304, the one or more circuits 1314, and external devices (e.g., non-contrast CT imaging tool).

In some embodiments, the one or more circuits 1314 may comprise hardware components. In other embodiments, the one or more circuits 1314 may comprise software components. The one or more circuits 1314 can comprise a segmentation circuit 1316 (e.g., a deep learning circuit) configured to perform a segmentation operation on one or more digitized images 108 to generate one or more segmented digitized images respectively identifying one or more regions of interest 110 (e.g., one or more of a heart, a liver, etc.). In some embodiments, the one or more segmented digitized images may comprise binary masks, which may be stored in the memory 1302.

In some additional embodiments, the one or more circuits 1314 may further comprise feature extraction circuit 1318 configured to extract a plurality of assessment features 118 from the one or more regions of interest 110 and/or from the ECG data 302. The plurality of assessment features 118 may be stored in the memory 1302.

In some embodiments, the one or more circuits 1314 may further comprise a machine learning circuit 1320 configured to operate one or more machine learning models (e.g., a Cox-proportional Hazard model) upon the plurality of assessment features 118 to generate a medical assessment 122.

Therefore, in some embodiments the present disclosure relates to an assessment system 100 a machine learning stage configured to generate a medical assessment using assessment features extracted from digitized imaging data and/or electrocardiogram data. Although the medical assessment is often described in terms of obstructive coronary artery disease, it will be appreciated that the disclosed method and apparatus may be applied to other areas where an imaging study can be used to decide on more advanced imaging (e.g., mammography versus breast MRI, sonography versus CT/MRI/PET, etc.)

In some embodiments, the present disclosure relates to a method that includes accessing data stored in an electronic memory, the data including digitized imaging data from a segmented non-contrast computerized tomography (CT) image of an obstructive coronary artery disease (OCAD) patient; extracting a plurality of assessment features from the data, the plurality of assessment features including image based features that characterize one or more of calcifications, fat tissue, heart structures, bone density, muscle, a lung, and breast tissue; and providing the plurality of assessment features and a plurality of clinical factors to a machine learning stage, the machine learning stage being configured to generate a medical assessment corresponding to whether or not the OCAD patient would benefit from additional diagnostic tests to identify a presence and extent of the obstructive coronary artery disease.

In other embodiments, the present disclosure relates to a method that includes accessing data stored in an electronic memory, the data including digitized imaging data and electrocardiogram (ECG) data corresponding to a patient; extracting a plurality of assessment features from the data, the plurality of assessment features including a plurality of image based assessment features extracted from the digitized imaging data and a plurality of ECG based assessment features extracted from the ECG data; and providing the plurality of assessment features and a plurality of clinical factors to a machine learning stage, the machine learning stage being configured to generate a medical assessment using the plurality of assessment features and the plurality of clinical factors

In yet other embodiments, the present disclosure relates to a non-transitory computer-readable medium storing computer-executable instructions that, when executed, cause a processor to perform operations, including accessing data stored in an electronic memory, the data including one or more regions of interest from a digitized image of a patient; extracting a plurality of assessment features from the one or more regions of interest; and providing the plurality of assessment features and a plurality of clinical factors to a machine learning stage, the machine learning stage being configured to generate a medical assessment for the patient corresponding to additional diagnostic imaging.

Examples herein can include subject matter such as an apparatus, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 113135).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing data stored in an electronic memory, the data comprising digitized imaging data from a segmented non-contrast computerized tomography (CT) image of an obstructive coronary artery disease (OCAD) patient;
extracting a plurality of assessment features from the data, wherein the plurality of assessment features comprise image based features that characterize one or more of calcifications, fat tissue, heart structures, bone density, muscle, a lung, and breast tissue; and
providing the plurality of assessment features and a plurality of clinical factors to a machine learning stage, wherein the machine learning stage is configured to generate a medical assessment corresponding to whether or not the OCAD patient would benefit from additional diagnostic tests to identify a presence and extent of the obstructive coronary artery disease.

2. The method of claim 1, wherein the plurality of assessment features characterize the fat tissue, the heart structures, and the calcifications.

3. The method of claim 1, wherein the medical assessment comprises a likelihood that a cardiac computed tomography angiography (CCTA) would yield a determination of obstructive disease.

4. The method of claim 1, further comprising:
accessing electrocardiogram (ECG) data corresponding to the OCAD patient from the electronic memory;
extracting the plurality of assessment features from the digitized imaging data and from the ECG data;
generating input data having values corresponding to the plurality of assessment features and the plurality of clinical factors; and
providing the input data to the machine learning stage.

5. The method of claim 1, wherein the medical assessment is a weighted mix of likelihoods that CCTA would characterize obstructive disease via a reporting system of Coronary Artery Disease Reporting and Data System (CAD-RADS) or fractional flow reserve (FFR).

6. The method of claim 1, wherein the medical assessment comprises a recommendation including one or more of performing one or more of a CCTA scan, a PET scan, a SPECT scan, an MRI scan, a stress ECG, active surveillance, or to do nothing.

7. The method of claim 6, wherein the machine learning stage is configured to generate a numeric value, the numeric value being compared to a threshold value to generate the medical assessment.

8. A method, comprising:
accessing data stored in an electronic memory, the data including digitized imaging data and electrocardiogram (ECG) data corresponding to a patient;
extracting a plurality of assessment features from the data, the plurality of assessment features including a plurality of image based assessment features extracted from the digitized imaging data and a plurality of ECG based assessment features extracted from the ECG data; and
providing the plurality of assessment features and a plurality of clinical factors to a machine learning stage, wherein the machine learning stage is configured to generate a medical assessment using the plurality of assessment features and the plurality of clinical factors.

9. The method of claim 8, wherein the medical assessment corresponds to whether or not the patient would benefit from one or more additional diagnostic tests to identify a presence and extent of obstructive coronary artery disease.

10. The method of claim 9, further comprising:
utilizing the one or more additional diagnostic tests to assess if the patient should undergo a revascularization procedure.

11. The method of claim 10, wherein the one or more additional diagnostic tests comprise a cardiac computed tomography angiography (CCTA).

12. The method of claim 8, wherein the plurality of ECG based assessment features include one or more of a duration of an interval, an area of a wave, an axis of an interval, an amplitude of a wave, and a heart rate of a full recording.

13. The method of claim 8, wherein the medical assessment is a risk of a coronary event within a predetermined time period.

14. The method of claim 8, further comprising:
generating a median beat from an ECG reading; and
extracting one or more of the plurality of ECG based assessment features from the median beat.

15. The method of claim 8, wherein the digitized imaging data comprises a computerized tomography (CT) calcium score image.

16. A non-transitory computer-readable medium storing computer-executable instructions that, when executed, cause a processor to perform operations, comprising:
accessing data stored in an electronic memory, the data including one or more regions of interest from a digitized image of a patient;
extracting a plurality of assessment features from the one or more regions of interest; and
providing the plurality of assessment features and a plurality of clinical factors to a machine learning stage, wherein the machine learning stage is configured to generate a medical assessment for the patient corresponding to additional diagnostic imaging.

17. The non-transitory computer-readable medium of claim 16, wherein the medical assessment comprises one or more of a likelihood that a cardiac computed tomography angiography (CCTA) would yield a determination of obstructive disease.

18. The non-transitory computer-readable medium of claim 16,
wherein the data further comprises electrocardiogram data; and
wherein the plurality of assessment features are extracted from the one or more regions of interest and the electrocardiogram data.

19. The non-transitory computer-readable medium of claim 18, wherein the plurality of assessment features comprise image based assessment features that characterize fat tissue, heart structures, and calcifications and ECG based assessment features extracted from the electrocardiogram data.

20. The non-transitory computer-readable medium of claim 18, wherein the electrocardiogram data is collected in a manner that is synchronized with obtaining the non-contrast computerized tomography image.
